(19) United States
(12) Patent Application Publication
(10) Pub. No.: US 2025/0143614 A1
(43) Pub. Date: May 8, 2025

(54) SYSTEMS AND METHODS FOR MULTI-MODAL STRESS TRACKING

(71) Applicants: UNIVERSITY OF MARYLAND, COLLEGE PARK, College Park, MD (US); GEORGIA TECH RESEARCH CORPORATION, Atlanta, GA (US)

(72) Inventors: Jin-Oh HAHN, College Park, MD (US); Yuanyuan ZHOU, College Park, MD (US); Jesse PARREIRA, College Park, MD (US); Yekanth Ram CHALUMURI, College Park, MD (US); Azin MOUSAVI, College Park, MD (US); Omer T. INAN, Atlanta, GA (US); Jesus Antonio SANCHEZ-PEREZ, Atlanta, GA (US); Asim H GAZI, Atlanta, GA (US); Anna HARRISON, Atlanta, GA (US)

(21) Appl. No.: 18/939,326

(22) Filed: Nov. 6, 2024

Related U.S. Application Data
(60) Provisional application No. 63/596,315, filed on Nov. 6, 2023.

(51) Int. Cl. A61B 5/16; A61B 5/0205; A61B 5/021; A61B 5/024; A61B 5/0245; A61B 5/11; A61B 5/352; G16H 40/63; G16H 50/20

(52) U.S. Cl. CPC A61B 5/165; A61B 5/0205; A61B 5/352; G16H 40/63; G16H 50/20; A61B 5/02125; A61B 5/02405; A61B 5/02416; A61B 5/0245; A61B 5/1102

(57) ABSTRACT

A system for identifying mental stress in a user is provided herein. A first sensor has at least one electrode configured to contact the user's skin to measure heart-based electrical signals of the user. A second sensor has at least one light emitter and at least one light sensor. A third sensor has at least one force sensor and can be configured to measure movement signals of the user's body reflecting cardio-mechanical activity of the user. The heart-based electrical signals, PPG signals, and movement signals in real time are received by a processor via at least one electrical input of the system. A set of mental stress signatures are continuously determined. The mental stress signatures are provided to an inference model to determine a probability of an acute mental stress state of the user. An output can indicate whether the user is presently in an acute mental stress state.

### CROSS-REFERENCE TO RELATED APPLICATION(S)

This application claims the benefit of U.S. Provisional Patent Application Ser. No. 63/596,315 filed on Nov. 6, 2023.

### STATEMENT OF GOVERNMENT SUPPORT

This invention was made with government support under N000142112031 awarded by the Office of Naval Research. The government has certain rights to the invention.

### TECHNICAL FIELD

The systems, methods, embodiments, and novel concepts discussed herein relate generally to health monitoring devices and applications. More specifically, certain examples described herein generally relate to systems, methods, devices, and applications that provide multi-modal stress tracking.

### BACKGROUND

Mental stress can affect both an individual's quality of life and wellbeing. Beyond simply a person's emotional state, mental stress can impact physiological systems within the human body, including profound implications on cardiovascular (CV) health. Mental stress is known to be detrimental to coronary artery disease (CAD) patients. In fact, it is a risk factor of CV disorders and adverse CV events in CAD patients. Chronic mental stress can cause atherosclerosis and hypertension, which may ultimately lead to CV disease such as CAD. Mental stress can even impact healthy individuals by causing transient endothelial dysfunction. Hence, the ability to continuously monitor and mediate (both acute and chronic) mental stress has the potential to improve psychophysiological health.

Acute mental stress elicits a multitude of physiological responses through changes in sympathetic and parasympathetic autonomic reflexes. However, current techniques suggest opportunities that have not been extensively explored. First, some prior work employed physiological signals sensed by hardware that is not convenient. For example, the electroencephalogram (EEG) is a frequently utilized physiological signal for acute mental stress monitoring via surface-level brain activities. However, its sensing requires a large number (typically >16) of electrodes placed on the scalp, which, although technically non-invasive, is far from being convenient and practical. Such a modality may be useful only in controlled laboratory and high-resource settings. Second, prior work has focused predominantly on the use of individual physiological signals in isolation. Of course, there are reports in which multiple physiological signals were used for acute stress monitoring. However, the majority of those studies still treated each physiological signal independently of each other (in that signatures of mental stress were derived from individual physiological signals, e.g., the electrocardiogram (ECG), EEG, skin conductance response (SCR), and skin temperature). In contrast, cross-integration of multi-modal physiological signals (e.g., leveraging fiducial points acquired from multiple modalities of physiological signals to derive signatures of acute mental stress) has not been rigorously explored. Third, prior work seldom employed cardio-mechanical signals, including the seismocardiogram (SCG) and the ballistocardiogram (BCG) in the context of acute mental stress monitoring. Prior work has demonstrated that in conjunction with other physiological signals, e.g., ECG and the photoplethysmogram (PPG), SCG and BCG have potential in monitoring blood pressure (BP) and other cardiovascular (CV) parameters.

Despite the existence of multiple sensing modalities relating to CV parameters, their use in accurate interpreting mental stress states has not been realized. To the extent any wearable or non-invasive CV sensing modalities can be said to indicate “stress” in a user, such indicates are largely inaccurate and do not distinguish between physical stress, mental stress, emotional stress, etc. This is due, in part, to the fact that single, univariate sensing is necessarily limited in what can be intuited from its measurements. Therefore, a need exists for a wearable, comfortable, non-invasive, and accurate system and method that can examine more complex, multivariate, interactions among signals from multiple sensing modalities to determine mental stress, specifically.

### SUMMARY

The following presents a simplified summary of one or more aspects of the present disclosure, in order to provide a basic understanding of such aspects. This summary is not an extensive overview of all contemplated features of the disclosure, and is intended neither to identify key or critical elements of all aspects of the disclosure nor to delineate the scope of any or all critical elements of all aspects of the disclosure nor to delineate the scope of any or all aspects of the disclosure. Its sole purpose is to present some concepts of one or more aspects of the disclosure in a simplified form as a prelude to the more detailed description that is presented later.

In some aspects, the present disclosure can provide a system for identifying mental stress in a user. A first sensor can have at least one electrode configured to contact the user's skin to measure heart-based electrical signals of the user. A second sensor can have at least one light emitter and at least one light sensor. The second sensor can measure photoplethysmogram (PPG) signals of the user. A third sensor can have at least one force sensor and can be configured to measure movement signals of the user's body reflecting cardio-mechanical activity of the user. A processor can be configured to receive the heart-based electrical signals, the PPG signals, and the movement signals in real time via at least one electrical input of the system. A set of mental stress signatures can continuously be determined, wherein at least one of the mental stress signatures is a composite signature determined from fiducial values derived from at least two of the heart-based electrical signals, the PPG signals, and the movement signals. The mental stress signatures can be provided to an inference model configured to determine a probability of an acute mental stress state of the user based on a probability density distribution associated with the mental stress signatures. An output can be generated that can be indicative of whether the user is presently in an acute mental stress state based on the probability.

In some aspects, the present disclosure can provide a method for tracking a user's mental stress state. Continuous physiological data can be obtained regarding the user during a measurement period. The physiological data can include data types including at least cardio-electrical data, cardio-mechanical data, and vascular data. At least heartbeat waveform information, heart valve opening information, and blood volume change information can be determined from the physiological data. A first set of mental stress signatures can be determined for a first time slice during the measurement period, from the heartbeat waveform information, heart valve opening information, and blood volume change information. At least one signature can be a multi-modality mental stress signature derived from more than one data type of the physiological data. The first set of mental stress signatures for the first time slice can be provided to a trained machine learning algorithm, to obtain probability indications of whether each first mental stress signature reflects a mental stress state and a no-mental stress state. A mental stress indication can be determined for the first time slice based on a weighted aggregation of the probability indications for each mental stress signature of mental stress state and no-mental stress state. A continuous mental stress score can be updated accordingly. The steps provided in the method can be performed subsequently for subsequent time slices during the measurement period to obtain mental stress indications for the subsequent time slices. The continuous mental stress score can be updated to account for the mental stress indication for the second time slice. The result can be outputted to a software application of a user device to provide the user with information relating to their mental stress states during the measurement period.

These and other aspects of the disclosure will become more fully understood upon a review of the drawings and the detailed description, which follows. Other aspects, features, and embodiments of the present disclosure will become apparent to those skilled in the art, upon reviewing the following description of specific, example embodiments of the present disclosure in conjunction with the accompanying figures. While features of the present disclosure may be discussed relative to certain embodiments and figures below, all embodiments of the present disclosure can include one or more of the advantageous features discussed herein. In other words, while one or more embodiments may be discussed as having certain advantageous features, one or more of such features may also be used in accordance with the various embodiments of the disclosure discussed herein. Similarly, while example embodiments may be discussed below as devices, systems, or methods embodiments it should be understood that such example embodiments can be implemented in various devices, systems, and methods.

### BRIEF DESCRIPTION OF THE DRAWINGS

FIG. 1 is a flowchart depicting an example protocol for obtaining training data, as utilized in certain experiments by the inventors.

FIG. 2 is a generalized diagram of placement locations for certain sensor types, according to one possible configuration in an experiment performed by the inventors.

FIG. 3 is a graph showing alignment of signals acquired by various modalities acquired during experiments performed.

FIG. 4 is a diagram conceptually illustrating process flow for a certain embodiments described herein.

FIG. 5 is a graph showing confidence levels and stress probabilities computed during experiments performed.

FIG. 6A-6F are graphs of mental stress and no-stress distributions.

FIG. 7A-7C are graphs of values recorded of mental stress signatures during experiments performed.

FIG. 8A-8B are graphs of results obtained during experiments performed, compared to time windows of confirmed mental stress states.

FIG. 9A-9B are graphs of results obtained during experiments performed, compared to time windows of confirmed mental stress states.

FIG. 10A-10B are graphs of mental stress and no-stress distributions.

FIG. 11 is a block diagram depicting an example component arrangement and data flow, in accordance with some aspects of the description herein.

FIG. 12 is a flow chart depicting an example process for identifying mental stress state, in accordance with aspects of the description herein.

FIG. 13 is a flow chart depicting example processes for generating specific types of mental stress predictors, in accordance with aspects of the description herein.

### DETAILED DESCRIPTION

The detailed description set forth below in connection with the appended drawings is intended as a description of various configurations and is not intended to represent the only configurations in which the subject matter described herein may be practiced. The detailed description includes specific details to provide a thorough understanding of various embodiments of the present disclosure. However, it will be apparent to those skilled in the art that the various features, concepts, and embodiments described herein may be implemented and practiced without these specific details. In some instances, well-known structures and components are shown in block diagram form to avoid obscuring such concepts. Likewise, while certain advantages of the systems and methods described herein are highlighted, it should be recognized that additional advantages may flow from use of these systems and methods even though not stated herein.

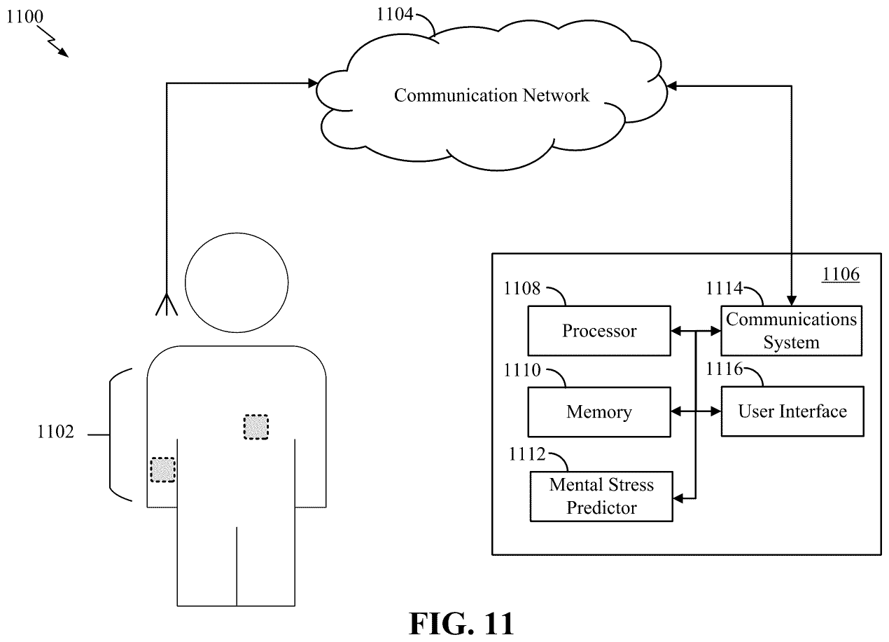

Referring now to FIG. 11, various configurations of hardware will be described, including processing resources, sensor modalities, and data flow among components.

FIG. 11 shows a block diagram illustrating an example of a system 1100 for identifying mental stress in a user using one or more physiological sensors in contact with the user's skin. In some examples, the system 1100 can utilize one or more sensors on a smart-watch, a phone, or other user device. In some examples, the system 1100 can utilize one or more electrodes attached on a user's body.

In some examples, a computing device 1106 can obtain physiological data from a sensor 1102 (such as a smart watch, electrode(s), etc.) or other connected device via a communication network 1104. In some examples, the physiological data 1102 can include cardio-electrical data, cardio-mechanical data, and/or vascular data (for ease of reference, the term “physiological data” will in some instances be used to refer to all of such data types, as well as other potential data types that can be non-invasively determined from a human body).

As depicted, the sensor 1102 can comprise one or more electrodes place on a user's body. As will be understood from the description herein, the sensor 1102 may be a standalone sensor, or may be a variety of types of sensors. For example, sensor 1102 may be a ballistocardiograph (BCG) sensor, a seismocardiography (SCG) sensor, a heart-band, an electrode patch, a watch, or a similar device used to obtain physiological data.

The computing device 1106 can include a processor 1108. In some embodiments, the processor 1108 can be any suitable hardware processor or combination of processors, such as a central processing unit (CPU), a graphics processing unit (GPU), an application specific integrated circuit (ASIC), a field-programmable gate array (FPGA), a digital signal processor (DSP), a microcontroller (MCU), cloud resource, etc.

The computing device 1106 can further include, or be connected to, a memory 1110. The memory 1110 can include or comprise any suitable storage device(s) that can be used to store suitable data (e.g., physiological data, a mental stress prediction model, etc.) and instructions that can be used, for example, by the processor 1108. The memory may be a memory that is “onboard” the same device as the sensor that detects the physiological data, or may be a memory of a separate device connected to the computing device 1106. Methods for identifying and/or predicting mental stress in a user based on data received from sensor 1102 may operate as independent processes/modules on the computing device 1106, such as on a separate mental stress predictor engine 1112 that runs on the same processor 1108 or a specialty processor (such as a GPU) that achieves greater efficiency in processing the physiological data. The memory 1110 can include any suitable volatile memory, non-volatile memory, storage, or any suitable combination thereof. For example, memory 1110 can include random access memory (RAM), read-only memory (ROM), electronically erasable programmable read-only memory (EEPROM), one or more flash drives, one or more hard disks, one or more solid state drives, one or more optical drives, etc.

In further examples, computing device 1106 can receive or transmit information (e.g., receiving physiological data from sensor 1102, transmitting instructions to sensor 1102, or transmitting predictions or stress reports to remote devices, etc.) and/or any other suitable system over a communication network 1104. In some examples, the communication network 1104 can be any suitable communication network or combination of communication networks. For example, the communication network 1104 can include a Wi-Fi network (which can include one or more wireless routers, one or more switches, etc.), a peer-to-peer network (e.g., a Bluetooth network), a cellular network (e.g., a 3G network, a 4G network, a 5G network, etc., complying with any suitable standard, such as CDMA, GSM, LTE, LTE Advanced, NR, etc.), a wired network, etc. In one embodiment, communication network 1104 can be a local area network, a wide area network, a public network (e.g., the Internet), a private or semi-private network (e.g., a corporate or university intranet), any other suitable type of network, or any suitable combination of networks. Communications links shown in FIG. 11 can each be any suitable communications link or combination of communications links, such as wired links, fiber optic links, Wi-Fi links, Bluetooth links, cellular links, etc.

In further examples, computing device 1106 can further include a user interface 1116. In one embodiment, the interface 1116 can include any suitable display devices, such as a watch face/screen, a cell phone screen, a computer monitor, a touchscreen, a television, an infotainment screen, etc. to display a prediction or report of identified mental stresses and/or to guide a user through processes for generating training data.

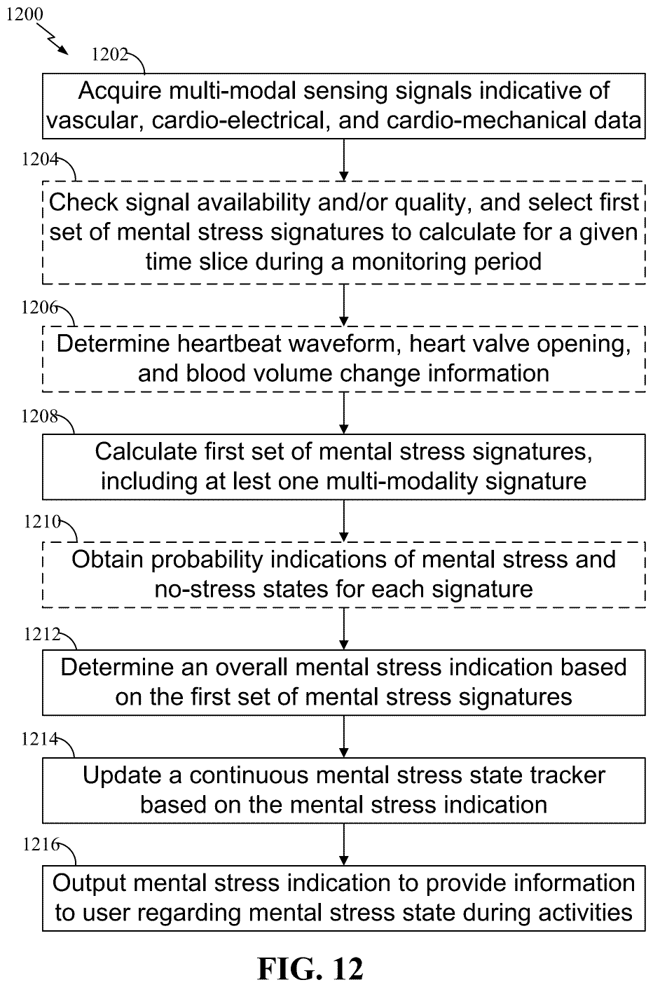

Referring now to FIG. 12, an example process 1200 is depicted for determining, characterizing, and/or tracking a user's state of mental stress during a measurement period (e.g., during a given hour, day, week, month, etc.). As described further below, process 1200 may generate individual, periodic, or continuous information regarding a user's state of mental stress (e.g., including stress scores, stress characterization, indications of stress states, etc.) that can be leveraged in several different applications for aiding individuals in managing mental stress in real-time, instantaneous situations, as well as over longer periods of time.

At block 1202, process 1200 may entail acquiring multi-modal sensing signals indicative of vascular, cardio-electrical, and cardio-mechanical data. Multi-modal sensing signals may be acquired from an individual device having multiple different types of sensors, from multiple individual sensing devices that transmit signals of different modalities to a processing device, or other configurations in which sensors obtain signals indicative of cardiovascular information of a subject's body (e.g., non-invasive surface sensors) through different forms of measurement providing independent information from one another. In one embodiment, sensing modalities may include photoplethysmography (PPG) for vascular signals, electrocardiogramar cardio-electrical signals, and seismocardiography (SCG) or ballistocardiography (BCG) for cardio-mechanical signals. Alternative embodiments may employ additional sensors, such as electrodermal activity (EDA) sensors or respiratory effort sensors, to capture complementary data that may enhance stress detection accuracy. Signal acquisition may occur continuously or at intervals, depending on system constraints and the desired temporal resolution.

At block 1204, process 1200 may optionally involve assessing the availability and quality characteristics of each acquired signal. This may include checking for noise, artifacts, or other disruptions in the data and applying quality thresholds to determine whether the signals meet the necessary criteria for accurate processing. In alternative embodiments, assessing signal quality characteristics could involve calculating metrics such as signal-to-noise ratio (SNR) or using machine learning algorithms to detect artifacts. Thus, process 1200 may determine whether a given sensing modality is viable to use for mental stress characterization, or whether the signatures to be calculated should be adjusted so that only signatures based on viable sensing are used. Additionally, if a sensing modality is available that can be used as a surrogate of another sensing modality (e.g., BCG utilized as a proxy for SCG, or aortic valve opening timing derived from impedance cardiography (ICG)), then certain process 1200 may determine whether compensation factors are available to be applied at block 1204 in order to promote accuracy in timing measurements from which certain signatures are derived.

In one example, where BCG may be used instead of SCG to capture aortic valve opening or other heart valve timing information, a calibration factor may be applied. In some instances corresponding/overlapping ECG or other cardio-electrical measurement of heartbeat waveform may be utilized to calibrate BCG detection of AO to improve accuracy sufficient to be able to use BCG as a surrogate measurement of an SCG-based AO timing. If a calibration cannot be made or is not available, then process 1200 may determine at block 1204 that BCG cannot be utilized to measure AO or other heart valve opening timing.

In another example, if ICG signals were available, but SCG signals were not, then the impedance signal from an ICG sensor can be monitored to determine a fiducial inflection point (e.g., B-point) from which an AO timing could be approximated. However, again, this measurement may require compensation in order to be used reliably as a measure of AO for purposes of signature determinations in subsequent blocks. Thus, adjustment for respiratory artifacts and signal alignment with ECG may be needed. If these compensations are not available, then process 1200 may determine that ICG cannot be used as a proxy of an SCG-based AO measurement.

In another example, if ECG signals are not available or exhibit too much noise or other artifacts, then process 1200 may assess whether other sensing modalities can be utilized to determine heartbeat waveform information as a proxy of ECG. For example, PPG, BCG, ICG, and SCG signals may provide information relating to heart rhythms sufficient to inform at least some factors of a typical heartbeat waveform, such as an R-wave peak. In some examples, the available signals (PPG, BCG, ICG, and/or SCG) may be monitored in association with an ECG signal of a given user for a period of time, involving physical, mental, and emotional stress states. A machine learning algorithm (e.g., an RNN, LSTM, or GRU, or a CNN combined with an RNN, a temporal CNN, or an attentional-based transformer model) could be trained to monitor ECG signals simultaneously with other available signals (e.g., PPG and BCG), to begin to learn which features of the available signals are indicative of the R-wave peak of an ECG signal, based on a given time window. Thus, process 1200 may determine whether such an algorithm is sufficiently accurate, trained, and current so as to be able to leverage non-ECG modalities to determine an R-wave peak, or whether only signatures that do not require ECG sensing should be used.

Based on the quality and availability assessment of block 1204, process 1200 may select a subset of the available signals to prioritize high-quality data for further analysis.

At block 1206, process 1200 may process the raw multi-modality sensing signals to extract cardiac and vascular parameters that may be predictive of mental stress states, such as determining key peaks and cycles of the heartbeat waveform from ECG data, heart rate variability data, heart valve opening events from SCG or BCG data, and blood volume changes from PPG data. This may include identifying fiducial points, such as the R-wave peak in ECG or the aortic valve opening (AO) point in SCG or BCG. Alternative embodiments may also utilize different algorithms for feature extraction, such as certain filtering techniques or statistical techniques (e.g., if measurement of a signature would occur over a time window comprising more than one heartbeat), to improve accuracy across varying physiological and environmental conditions.

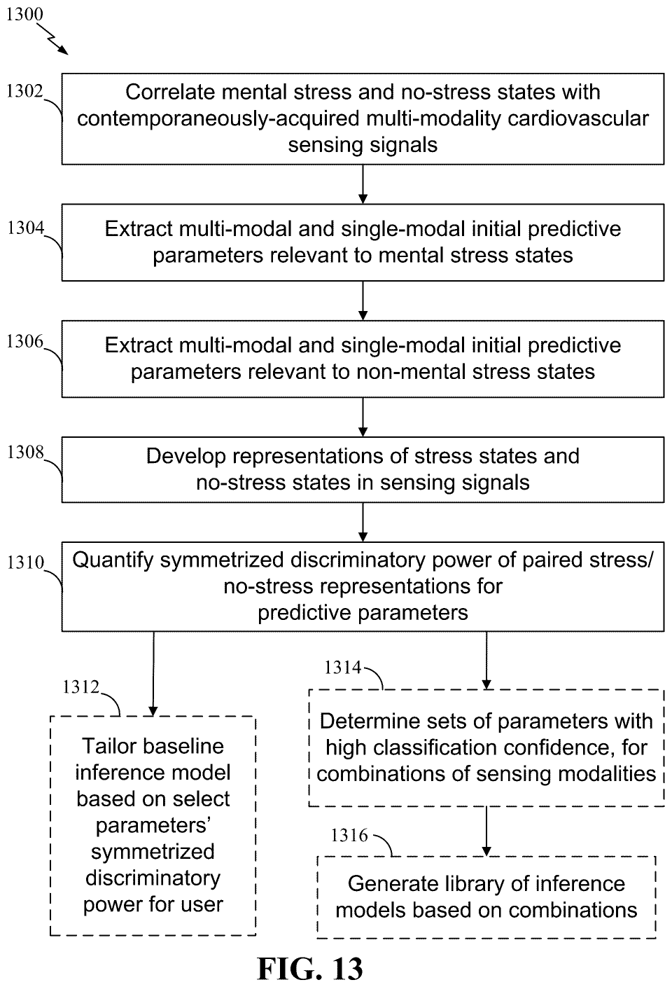

At block 1208, process 1200 may calculate a first set of mental stress signatures, which may include at least one multi-modality signature. Each mental stress signature may comprise a value or parameter from one of the sensing modalities (e.g., a single-modality parameter that is determined to be relevant to mental stress state, such as through clinical studies or user-specific training) or may comprise a multi-modality parameter that is calculated from values of more than one sensing modality. For example, pulse arrival time (PAT) may be a mental stress signature, and a value for this signature may be calculated as a composite of certain aspects derived from both ECG and PPG signals, or the pre-ejection period (PEP) may be determined using multiple data types, such as ECG and SCG data. In alternative embodiments, different combinations of parameters, including these and other signatures, such as heart rate variability (HRV) or skin conductance response (SCR), may be employed to calculate the first set of mental stress signatures, depending on available sensors and desired specificity. The selected signatures may reflect physiological changes associated with mental stress and may be derived through single-signal processing or cross-integration of multiple signals, or both. FIG. 13 and the Examples section below provides further information on selection of signatures that may be indicative of a mental stress or no-stress state.

At block 1210, process 1200 may optionally involve determining probability indications for mental stress and no-stress states associated with each calculated mental stress signature. In one embodiment, a probabilistic model may be applied using probability density distributions for stress and no-stress states based on historical or training data. To enhance accuracy, weights may be applied to these probabilities using a symmetric probability divergence score, such as Kullback-Leibler (KL) divergence, to quantify how strongly each signature supports a stress or no-stress state. Alternative embodiments could utilize machine learning models, such as Gaussian mixture models or neural networks, to estimate these probabilities based on the observed data patterns.

At block 1212, process 1200 may aggregate the individual probability indications from each stress signature of the first set of mental stress signatures to determine an overall mental stress indication for the point in time or time window from which the values of the signatures were calculated. This may involve a weighted summation, a voting mechanism, or other statistical combination of probabilities where each signature contributes to the overall mental stress indication based on its probability score and predictive power (e.g., divergence score). In some embodiments, process 1200 may apply a threshold to the aggregated probability score to classify the overall state as “stress” or “no-stress.” Alternative methods, such as ensemble averaging or Bayesian inference, could also be used to enhance robustness in diverse conditions.

At block 1214, process 1200 may update a continuous mental stress state tracker (e.g., a graph, histogram, or other visual indicator, a time-series, etc.) based on the overall mental stress indication for the given time period or time window. This tracker may maintain a time-series log of mental stress probabilities (overall stress state, stress state, no-stress state, or a combination thereof), a time-series log of just the mental stress indications that were determined, or other data storage approaches, allowing for both real-time actions to be triggered as well as trend analysis over time. In one embodiment, process 1200 may implement an averaging function, or a sliding window approach, to account for transient fluctuations in mental stress states, ensuring that short-lived stress indications (which may either be noise or simply too brief to be relevant to an individual's mental wellbeing) signatures do not overly influence the tracker. In alternative embodiments, the tracker could incorporate decay functions or moving averages to balance sensitivity and stability, providing more consistent feedback on stress levels during prolonged monitoring.

In yet further embodiments, the tracker may also store additional environmental, activity, location, context, and other information that may be relevant to the cause of the mental stress. For example, text message viewing, email receipt, media consumption, phone calls, locations, contacts, activities, calendar appointments, classes, meetings, and the like can be determined from user devices and accounts, and temporally associated with stress states.

At block 1216, process 1200 may output the mental stress indication to provide real-time or near-real-time information to the user. This output may include a visual or auditory alert, a percentage likelihood of being in a stress state, or a confidence score. In other embodiments, the output may be integrated into a wearable device interface or mobile application that displays stress levels graphically. In alternative embodiments, process 1200 may store stress data for retrospective analysis or for sharing with health professionals, potentially supporting interventions or tailored stress management recommendations. In other embodiments, the mental stress indication may be provided as a trigger to software applications on devices associated with the user (in some cases instead of actually informing the user of their stress state, which may be detrimental, or in addition to informing the user) to serve as a trigger of other functions directed to the user's wellbeing and health.

Referring now to FIG. 13, an example of a process design 1300 is depicted for developing, refining, and/or selecting predictive parameters and inference models that can be used to identify, characterize, and/or track mental stress and no-stress states from multi-modality cardiovascular sensing signals. As described further below, process 1300 may be used to generate user-specific inference models that are tailored from population/general models and/or inference models that utilize various combinations of sensing modalities and associated parameter sets (to allow for continuous monitoring even when some sensing types are not available or temporarily unsuitable for us). These models may be utilized in systems and processes such as described above with respect to FIGS. 11 and 12 that enable accurate, real-time stress detection across various contexts.

At block 1302, process 1300 may entail correlating mental stress and no-stress states with contemporaneously acquired multi-modal cardiovascular sensing signals. For example, a training data set may be received with labeled periods of confirmed mental stress and no-stress states for a given individual or population and/or training data may be acquired for a given user through a user interface that causes controlled mental stress/no-stress states (e.g., as described below in the Examples section) such as through a smartphone app. This may include collecting labeled datasets where specific periods of confirmed mental stress or no-stress states are matched with corresponding data contemporaneously acquired (e.g., acquired for the same pulse cycle or set of pulse cycles) from various sensing modalities, such as ECG, PPG, SCG, BCG, or other modalities mentioned herein. In some embodiments, additional signals, like electrodermal activity (EDA) or respiratory rate, may also be incorporated if available. Data collection may be continuous or occur at predetermined intervals, with labeled mental stress and no-stress states being based on objective or subjective assessments (e.g., self-reports, physiological benchmarks, or environmental triggers). In some embodiments, the users who generate the labeled datasets may be required to undergo mental stress in periods of time in which there was no physical or emotional stress (which can cause different or additional impacts on cardiovascular sensing) and/or in periods of time in which there was physical stress, emotional stress, or both. Thus, data can acquired and labeled to provide a robust basis on which to train inference models on factors specific to mental stress, and improve the ability of the models to distinguish mental stress from other types of stress.

At block 1304, process 1300 may involve developing representations of mental stress and no-stress states in the sensing signals. For example, process 1300 may utilize feature extraction methods to generate a set of digital signatures or statistical features that characterize the physiological responses associated with stress and no-stress conditions. In some embodiments, the inventors have determined that it is beneficial to develop these representations in terms of specific parameters that are known to be indicative of or related to mental stress, specifically, rather than allowing the models to learn from any/all features of the sensing signals. In some embodiments these parameters may include (or be exclusively limited to) cardiovascular parameters that primarily or substantially reflect or relate to changes due to cognitive demand or mental workload. For example, these representations may include specific sensing signal features, fiducial points, and parameters (e.g., R-wave amplitude, PPG signal amplitude, heart rate variability, heart rate complexity, SBP reactivity, vascular resistance, peripheral blood flow), timing intervals (e.g., pulse arrival time, pre-ejection period, pulse transit time, ejection times), or complex multi-modal combinations thereof (e.g., cardiac output (CO), stroke volume, respiratory arrhythmias. In some embodiments, a set of possible features, fiducial points, and parameters may be determined from a list of non-invasive measurements that can be taken from wearable devices, which may fall into categories of cardiac and vascular states such as diastolic (DP) and systolic (SP) blood pressure; stroke volume (SV); total peripheral resistance (TPR); heart rate variability (HRV); high/low frequency changes in HRV; cardio output (CO); pulse transit time (PTT) and pulse arrival time (PAT) (which can be inversely proportional to DP and SP); pre-ejection period (PEP), left ventricular ejection time (LVET) and PEP/LVET (which are inversely proportional to SV); PPG amplitude (which is inversely proportional to TPR); heart rate and HR/PEP and HR/(PEP/LVET) (which are conceptually equivalent to the product of HR and SV, i.e., CO), respectively); respiratory sinus arrhythmia; peripheral vascular resistance; electrodermal activity; skin conductance; vascular tone; ECG waveform changes such as in QRS complex; QT interval; etc. In some embodiments, dimensionality reduction techniques (e.g., principal component analysis) may be applied to reduce the feature set, retaining only the most informative representations of each state. Other embodiments may utilize known parameters plus parameters that may be learned specific to a given individual or population, such as through a machine learning process as described below.

At block 1306, process 1300 may quantify the symmetrized discriminatory power of paired stress/no-stress representations for predictive parameters. In one embodiment, process 1300 may apply a metric such as symmetrized Kullback-Leibler (KL) divergence, other divergence scoring methods, or other statistical distance measures to assess how distinct each parameter's distribution is between mental stress and no-stress states. Higher discriminatory power suggests that the parameter reliably distinguishes between these states.

At block 1308, process 1300 may extract multi-modal and single-modal initial predictive parameters relevant to mental stress states. Based on the discriminatory power quantified in block 1306, process 1300 may identify a set of parameters from the multi-modal data that are most indicative of mental stress states. Thus, process 1300 may identify a set or subset of parameters that are most predictive of mental stress state (or rank the available parameters) to serve as mental stress signatures. These parameters may include individual features (e.g., PPG amplitude), relative interactions between combinations of features across different sensing modalities (e.g., increases in a given vascular measurement without a corresponding increase in another cardiovascular measurement which could indicate another type of stress), and/or composite parameters derived from more than one sensing modality (e.g., pulse arrival time derived from ECG and PPG). This selection process may be flexible, allowing for alternative models to be generated that extract different parameter sets based on available data and user-specific characteristics.

At block 1310, process 1300 may similarly extract multi-modal and single-modal initial predictive parameters relevant to non-mental stress states. By identifying features that are characteristic of no-stress states, process 1300 can create a complementary set of parameters to further refine stress detection accuracy by determining confidence levels for both mental stress and no-stress states for a given time point or time window. Alternatively, extraction of parameters relevant to mental no-stress states could be done as part of block 1308 in conjunction with and/or at the same time that parameters relevant to mental stress states are extracted.

These no-stress parameters may also be used as a baseline or control in subsequent inference model development, such as tailoring a model to a given user, helping to distinguish normal physiological variations from stress-specific responses.

From these extracted parameters of mental stress and no-stress parameters at block 1308-1310, sets of mental stress signatures can be developed to serve as the basis of mental stress inference models. In some examples, these inference models may be based on large-population averages, specific population subtypes, or specific users. Thus, either user-specific or population/baseline models may be generated.

At block 1312, process 1300 may optionally update or tailor a baseline inference model (either a population-based general model or a preexisting user-specific model) based on the symmetrized discriminatory power of selected parameters for an individual user. For example, if the approaches of blocks 1302-1310 are performed and a set of parameters are determined, with associated information concerning discriminatory power, based solely on data for a given user, then the overall mental stress indication output determination steps (e.g., in FIG. 12) for an inference model can be adjusted to more closely reflect how mental stress manifests in the cardiovascular measurements of that user specifically. This tailoring or updating process may involve adjusting model weights or thresholds to account for user-specific responses to stress, as determined by the parameter distributions identified in blocks 1306, 1308, and 1310. In some embodiments, machine learning algorithms may be used to optimize the inference model based on these user-specific characteristics, incorporating adaptive elements that refine the model over time. In some instances, where the divergence score for a given parameter measured via a user-specific set of blocks 1302-1310 differ from the divergence score for a given parameter of the baseline model, the confidence/divergence information utilized in the model can be proportionately increased or decreased (or adjusted up to a threshold amount, such as no more than 10% change, 20% change, etc.) so that final mental stress indications match that person's response more closely. Also, if a given parameter that is not present in a baseline model is determined to have a threshold level of discriminatory power for a give user, that parameter can be added to the ensemble of mental stress signatures in the baseline model.

At block 1314, process 1300 optionally may generate a library of inference models based on various combinations of sensing modalities. This step may involve creating a variety of models that each rely on different combinations of the selected parameters so that differing combinations of sensing modalities are correspondingly relied upon (e.g., models that use ECG and PPG vs. models that use SCG and EDA). This model library enables flexible deployment across varying device configurations and environmental conditions.

At block 1316, process 1300 may optionally determine sets of parameters with high classification confidence for each combination of sensing modalities. This step involves evaluating the inference models from block 1314 to identify the parameter sets that yield the highest classification confidence for each available modality combination. This may include calculating accuracy, precision, and recall metrics, as well as examining model confidence scores. In some embodiments, process 1300 may apply threshold adjustments or weighting schemes to emphasize parameters with consistently high classification confidence. In some embodiments, inference models resulting from process 1300 may utilize ensemble methods, where multiple inference models are used in parallel to improve classification accuracy.

### Example Embodiments and Experimental Findings

In the following section, a discussion is presented of specific experiments performed by the inventors using particular embodiments contemplated herein. It is to be understood, however, that the scope of the present disclosure is not limited by the approaches the inventors took in performing their experiments. Rather, the experiments described below are meant merely to illustrate and validate all of the configurations, embodiments, and alternatives disclosed herein.

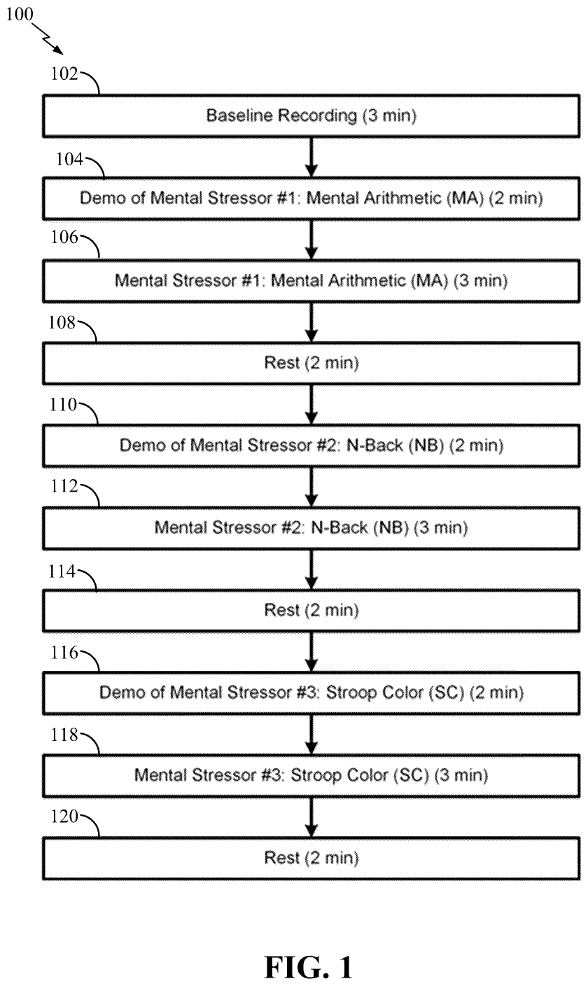

We acquired multiple modalities of physiological signals from 27 young healthy volunteers (age 19-30 years old; gender 17 male and 10 female; height 155-201 cm; weight 52-113 kg) under the approval of the Institutional Review Board (IRB) at the University of Maryland (Approval #813845, July 2020) and the Navy Human Research Protection Office (HRPO) as well as written informed consent. The participants underwent 3 acute mental stress interventions (FIG. 1). We provided a demo period before each acute mental stress intervention to familiarize the participants with the interventions. We selected the duration of the acute mental stressors and the rest periods based on the acute BP responses observed during our internal experiments: 3-5 min of acute mental stressors quickly raised BP (<1 min) and sustained the increase throughout the stressors, while removing the mental stressors quickly lowered BP (<1 min). We employed stressor duration in our work of 2 min demo+3 min stressor=5 min. We strictly followed the IRB guidelines to ensure the safety of study participants and staffs against novel coronavirus disease (COVID-19) infection. During the course of the experiment, we asked the participants to sit on a stool with minimal movement. We administered all the acute mental stressors using a custom-designed graphical user interface program with voice recognition to determine the correctness of the answers, which allowed the study staffs to exercise social distancing against COVID-19 infection.

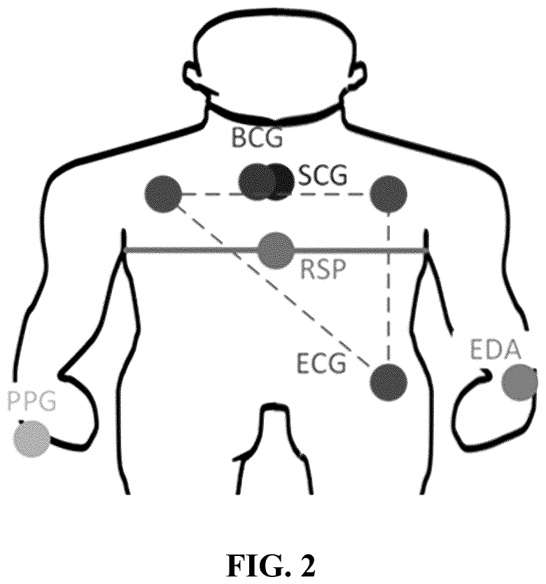

We measured 6 modalities of physiological signals during the experiment (FIG. 2), including (i) the chest ECG (BN-EL50 electrodes and BN-RSPEC-T transmitter, Biopac Systems, Goleta, CA, USA; representing cardio-electrical physiology); (ii) a finger clip PPG (8000AA, Nonin Medical, Plymouth, MN, USA; representing vascular physiology); (iii) the SCG and the BCG as anterior-posterior and superior-inferior accelerations at the chest (356A32 accelerometer and 482C15 signal conditioner, PCB Piezotronics, Depew, NY, USA; both representing cardio-mechanical physiology); (iv) respiratory effort (TSD201 sensor and BN-RSPEC-T transmitter, Biopac Systems, Goleta, CA, USA; representing respiratory physiology); and (v) the EDA (E4, Empatica, Cambridge, MA, USA; representing sudomotor physiology). Note that we did not consider brain activity signals in this work, because they are not ideally suited to convenient measurement in the setting of everyday living. We interfaced all the wearable devices to a desktop computer using a data acquisition unit (MP150, Biopac Systems, Goleta, CA, USA; except E4 which was interfaced directly to the same desktop computer) to synchronously record all the physiological signals at 1 kHz sampling rate.

We used the experimental dataset corresponding to 24 participants, after excluding 3 participants due to corrupted physiological measurements.

Digital Signatures: From the experimental dataset, we derived a number of initial predictive parameters (i.e., candidate digital signatures) of acute mental stress by (i) pre-processing the physiological signals, (ii) extracting fiducial points in them, and (iii) deriving the candidate digital signatures.

First, we pre-processed the physiological signal waveforms to remove low-frequency wander (by high-pass filtering) and high-frequency noise (by low-pass filtering). We filtered the ECG by a bandpass filter with 0.5-40 Hz passband. We filtered the PPG by a bandpass filter with 0.4-8 Hz passband. We filtered the SCG and the BCG by a bandpass filter with 1-40 Hz passband. We filtered the respiratory effort signal by a bandpass filter with 0.07-1 Hz passband. We selected the passbands based on the knowledge of physiologically meaningful frequency contents in the signals as well as our own experimentation. Second, we localized and extracted the R waves in the ECG using the Pan-Tompkins method. Third, we used the ECG R waves to gate individual beats in the PPG, SCG, and BCG. Fourth, we assessed the quality of these beats. For the PPG, we used the signal amplitude (low-quality if >4 median absolute deviations) and the signal quality index tool provided in the PhysioNet Cardiovascular Signal Toolbox with a threshold of 0.4. For the SCG and the BCG, we likewise used the signal amplitude (low quality if >10 times the median amplitude), followed by a template-based signal quality index tool we developed after 10-beat moving average filtering. For the respiratory effort signal, we used a method based on the power spectral density and the autocorrelation of the signal which we previously developed. For the EDA, we used a method based on the amplitude and the rate of change of the signal. Fifth, we removed low-quality signal beats (or windows of pre-specified length in case of the EDA signal; details provided later in this section) from the development of the proposed algorithm.

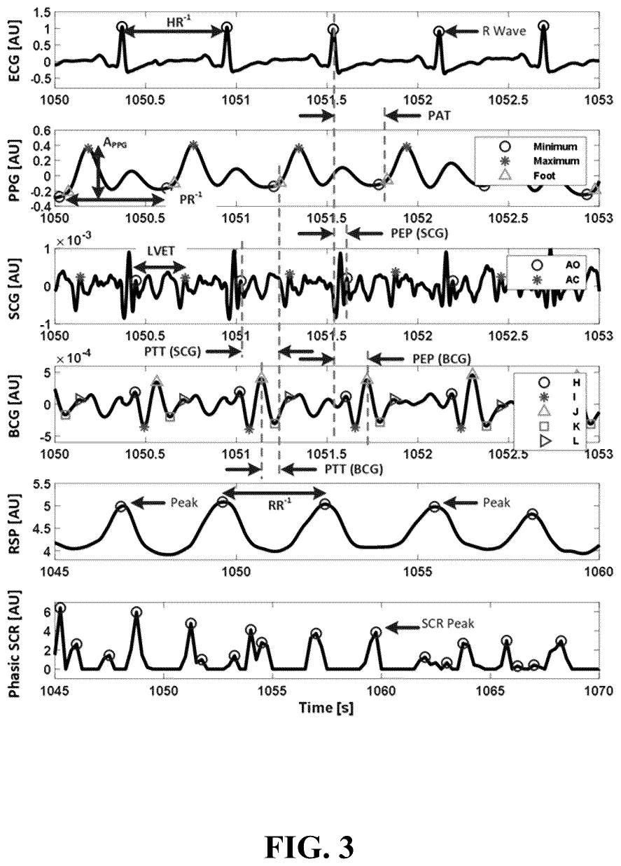

Second, we extracted fiducial points in the physiological signals on a cardiac beat-by-beat basis except the respiratory effort (on a respiratory breath-by-breath basis) and the EDA (on a window-by-window basis), as depicted in FIG. 3. From the PPG, we extracted the timings and values of its maximum and minimum peaks as well as its foot (using the intersecting tangent method). From the SCG, we extracted the timings of the AO point (a signature of aortic valve opening) and the AC point (a signature of aortic valve closure) as the prominent peaks in its early-(typically <100 ms after the ECG R wave) and end-systolic (typically 150-350 ms after the ECG R wave) wave complexes. From the BCG signal, we extracted its H, I, J, K, and L waves using a method based on a priori knowledge of the time interval between the ECG R wave and the BCG J wave (note that J wave is an approximate signature of aortic valve opening). From the respiratory effort signal, we extracted the timing of its maximum peak. From the sparse phasic component in the EDA signal, we extracted all the SCR peaks using a minimum peak height of 0.02 and minimum peak prominence of 0.3 as threshold based on a publicly available algorithm (cvxEDA).

Third, we derived candidate digital signatures of acute mental stress on an appropriate beat-by-beat, breath-by-breath, or window-by-window basis, as shown in FIG. 3. We derived HR and pulse rate as the reciprocal of the time interval between the ECG R waves and the PPG feet in the neighboring beats (i.e., current and its immediate previous beats), respectively. We derived PPG amplitude as the difference between its maximum and minimum values. We derived LVET as the time interval between AO and AC points in the SCG. We derived PEP as the time interval between (i) ECG R wave and SCG AO point and (ii) ECG R wave and BCG J wave (note that both SCG AO point and BCG J wave approximately represent aortic valve opening in this context). We derived PAT as the time interval between ECG R wave and PPG foot. We derived PTT as the time interval between (i) SCG AO point and PPG foot and (ii) BCG J wave and PPG foot (note that BCG J wave approximately represents aortic valve opening in this context). We derived respiratory rate (RR) as the reciprocal of the time interval between the maximum values in the neighboring breaths (i.e., current and its immediate previous breaths). We derived orienting response and its magnitude (NOR and MOR) as the number of SCR peaks and their average magnitude in each 25-second time window. We used a moving time window with an overlap of 24.75 seconds in deriving NOR and MOR. Using the signatures directly derived from the physiological signals, we defined a set of physiologically-inspired, approximate digital signatures intended to represent the collective CV state, including diastolic (DP) and systolic (SP) BP, stroke volume (SV), total peripheral resistance (TPR), HR, and cardio output (CO): PTT and PAT (which are inversely proportional to DP and SP), PEP and PEP/LVET (which are inversely proportional to SV), PPG amplitude (which is inversely proportional to TPR), HR, and HR/PEP and HR/(PEP/LVET) (which are conceptually equivalent to the product of HR and SV, i.e., CO), respectively. In this way, we derived a total of 17 candidate digital signatures of acute mental stress. However, we contemplated that additional candidate digital signatures of acute mental stress could have been derived as well, depending upon available sensing signals and study participants' tolerance for additional or potentially redundant measurements.

To account for the inter-individual variability in the absolute values of the digital signatures, we normalized all the candidate digital signatures using the z-score normalization on a participant-by-participant basis. Then, we smoothed all the candidate digital signatures by moving-average filtering with a sliding time window of 10 s and an incremental step of 1 s (which means that the candidate digital signatures were resampled at 1 Hz).

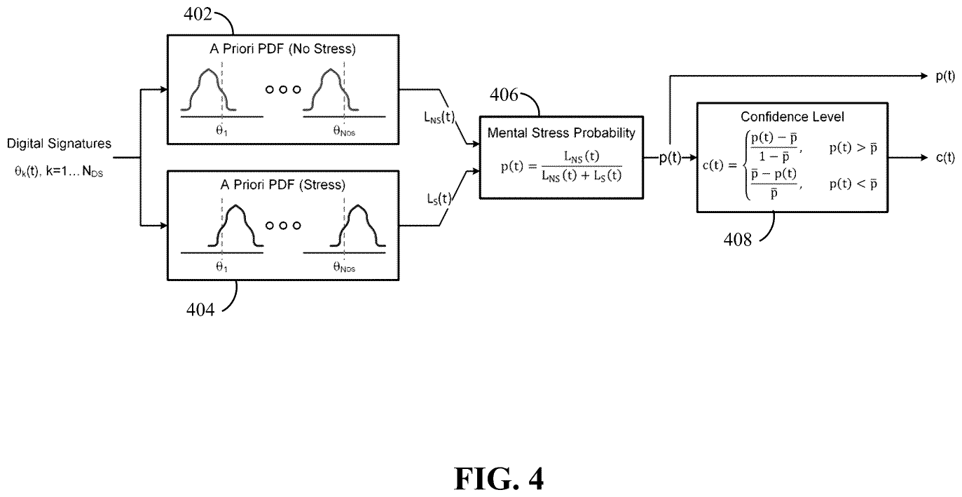

Mental Stress Detection: FIG. 4 provides a high-level overview of the example algorithm we used for detecting acute mental stress using the measurements of digital signatures. First, it receives a set of prominent digital signatures of acute mental stress. Second, it assesses the likelihood value of each digital signature pertaining to no stress and stress states using its a priori probability density functions pertaining to no stress and stress states. Third, it aggregates the likelihood values using weights proportional to the degree of discrepancy in the probability density functions between no stress and stress states (in terms of the symmetrized divergence). Fourth, it infers the “stress probability” (which means the probability with which a subject is subject to acute mental stress) and the corresponding confidence level (which means the degree of certainty in its inference) (see below for further discussion). Fifth, it detects the presence of acute mental stress if the stress probability is >0.5.

We developed this example of an acute mental stress detection algorithm as follows. First, we determined a set of prominent digital signatures of acute mental stress to be incorporated into our algorithm as well as their a priori probability density distributions via a preliminary leave-one-subject-out analysis. In this experiment, we tended toward using candidate digital signatures with known correlation to acute mental stress, though we also contemplate using machine learning methods to generate additional digital signatures. For each test participant, we (i) derived a priori probability density distributions of all the candidate digital signatures using the dataset of the remaining 23 participants; (ii) calculated the symmetrized divergences pertaining to all the candidate digital signatures; and (iii) determined prominent digital signatures based on the ranking of the candidate digital signatures. Then, we determined a set of prominent digital signatures to be used commonly in the evaluation. Second, we developed computational procedures to calculate the probability of acute mental stress and its confidence level.

Inference of A Priori Distributions of Candidate Digital Signatures: The proposed algorithm shown in FIG. 4 infers the probability of acute mental stress and its confidence level based on a priori knowledge of the probability density distributions associated with the digital signatures of acute mental stress. In this work, we used a collective variational inference (C-VI) method to derive the probability density functions, which is suited to the inference of cohort-level characteristics from subject-level datasets. We modeled the measurement of a digital signature as the sum of the true digital signature and measurement noise (due to, e.g., imperfect extraction of fiducial points):

$${y}_{i k} ( t ) = {θ}_{ik} ( t ) + {n}_{k} ( t ) \qquad ( 1 )$$

where yᵢₖ(t) is the observed k^{th} digital signature in the i^{th} subject at a time instant t, θᵢₖ(t) is the corresponding true (i.e., noise-free) digital signature, and nₖ(t) is the measurement noise (note that it depends only on the index k because it is assumed to depend only on the sensor). We assumed that θᵢₖ(t) originates from its cohort-level probability density [symbol] : θᵢₖ(t)˜ [symbol] (ϕₖ), where ϕₖ k are latent parameters characterizing [symbol] . In this work, we modeled [symbol] 's as Gaussian density functions. Hence, ϕₖ={mₖ, σₖ} where mₖ and σₖ are mean and standard deviation (SD) of the cohort-level probability density of [symbol] .

In a leave-one-subject-out analysis setting, we repeatedly derived a priori probability density distributions of all the candidate digital signatures listed herein. For each test participant, we analyzed the dataset of the remaining 23 participants using the C-VI method to derive the cohort-level probability density functions of all the candidate digital signatures pertaining to no stress and stress states. In the C-VI method, we used the dataset of initial baseline recording, 3 rest periods, and the first half of 3 demo periods (see FIG. 1 for a depiction of the recording protocol) to derive probability density functions pertaining to no stress state: [symbol] ={ [symbol] (ϕ_{NS,k})}_{k=1 . . . 17}. We used the dataset of 3 acute mental stress interventions and the second half of 3 demo periods (again, as shown in FIG. 1) to derive probability density functions pertaining to stress state: [symbol] ={ [symbol] (ϕ_{S,k})}_{k=1 . . . 17}. In both cases, we inferred the latent parameters pertaining to all the candidate digital signatures (that is, ϕ_{NS,k}={m_{NS,k}, σ_{NS,k}} for no stress state and ϕ_{S,k}={m_{S,k}, σ_{S,k}} for stress state). In this way, we derived 24 sets of a priori probability density functions pertaining to all the candidate digital signatures, which were analyzed in Section 2.3.2 to select prominent digital signatures to be incorporated into our acute mental stress detection algorithm.

Selection of Prominent Digital Signatures: Using the 24 sets of a priori probability density functions pertaining to all the candidate digital signatures, we selected a set of prominent digital signatures of acute mental stress to be incorporated into our acute mental stress detection algorithm for this particular experiment. To accomplish this, we quantitatively compared a priori probability density functions of the digital signatures between no stress vs stress states as follows. First, we repeatedly (i.e., 24 times) ranked the 17 candidate digital signatures in descending order in terms of symmetrized divergence associated with the cohort-level probability density functions between stress vs no stress states. Here, symmetrized divergence pertaining to a digital signature is beneficial to quantify the degree of discrepancy between the two states we are concerned with: no stress vs stress states pertaining to the digital signature in terms of probability density function. Of course, a more granular set of states could also be used, such as high stress, low stress, medium stress, no-stress, etc. Second, we selected prominent digital signatures to be incorporated into our algorithm based on the 24 digital signature rankings in descending order of symmetrized divergence through hard voting. The rationale was to detect acute mental stress by exploiting those digital signatures which assume largely distinct values between no stress vs stress conditions.

We determined the number of digital signatures to be incorporated into our algorithm as follows. We defined the optimal number of prominent digital signatures as the minimum number of digital signatures in our algorithm beyond which the classification accuracy is improved only marginally (<0.5%). Of course, other factors could also be taken into account, such as signal quality characteristics, signal reliability, availability of sensing modalities, system constraints, etc. To determine this value, we repeated a leave-one-subject-out analysis as follows while increasing the number of candidate digital signatures employed in the algorithm from 1 to 17. For each test participant, we calculated the symmetrized divergences pertaining to all the candidate digital signatures using a priori probability density distributions derived using the dataset of the remaining 23 participants. Using these symmetrized divergence values, we calculated the weights αₖ's in Eq. (4). Then, we inputted the time series sequence of candidate digital signatures pertaining to the test participant on a sample-by-sample basis to their respective a priori probability density distributions pertaining to no stress vs stress states to calculate L_{NS}(t) and L_{S}(t) in Eq. (3). Then, we calculated the probability of acute mental stress p(t) in Eq. (2) and its confidence level c(t) in Eq. (5). We performed the leave-one-subject-out analysis described above for each participant (i.e., 24 times in total). Then, we determined the optimal number of prominent digital signatures guided by the classification accuracy associated with the 17 acute mental stress classifiers. Then, we incorporated the optimal number of high-ranking (i.e., prominent) digital signatures into our acute mental stress detection algorithm.

Inference of Acute Mental Stress Probability and Confidence Level: The example algorithm we used for detecting acute mental stress in our experiment determines acute mental stress state every time new measurements of digital signatures become available (i.e., at 1 Hz interval, though other frequencies of measurement could also be utilized, whether in a predetermined or dynamic manner (e.g., during high stress and/or physical stress states, or other scenarios in which heart rate is elevated, a faster frequency of measurement may be utilized, whereas in other states a much lower frequency of measurement may be utilized such as when a user is sleeping, meditating, etc.), by evaluating the “aggregated” likelihoods with which the digital signatures belong to their a priori probability density distributions pertaining to no stress vs stress states (FIG. 4). If the digital signatures are more likely to belong to a priori distribution pertaining to no stress state than stress state, the algorithm infers that the subject is not in an acute mentally stressed state. However, if the digital signatures are more likely to belong to a priori distribution pertaining to stress state than no stress state, the algorithm infers that the subject is in an acute mentally stressed state.

To quantitatively determine whether a subject is under acute mental stress or not, we developed two novel continuous metrics: (i) probability of acute mental stress (which indicates the degree of certainty with which a subject is under acute mental stress: 0 if the subject is certainly not under acute mental stress and 1 if the subject is certainly under acute mental stress) and (ii) confidence level associated with the probability of acute mental stress (which indicates the degree of certainty regarding the probability of acute mental stress). First, we developed the probability of acute mental stress p(t) at a time instant t as follows:

$$p ( t ) = \frac{{L}_{N S} ( t )}{{L}_{N S} ( t ) + {L}_{S} ( t )} \qquad ( 2 )$$

where L_{NS}(t) and L_{S}(t) are the weighted aggregation of the negative log likelihoods associated with all the digital signatures inputted to the algorithm at the time instant t:

$${L}_{N S} ( t ) = {\sum }_{k = 1}^{{N}_{DS}} {α}_{k} {L}_{N S , k} ( t ) = {\sum }_{k = 1}^{{N}_{DS}} {α}_{k} [ \frac{1}{2} {( \frac{{θ}_{k} ( t ) - {m}_{N S , k}}{{σ}_{N S , k}} )}^{2} + log \sqrt{2 π} {σ}_{NS , k} ] {L}_{S} ( t ) = {\sum }_{k = 1}^{{N}_{DS}} {α}_{k} {L}_{S , k} ( t ) = {\sum }_{k = 1}^{{N}_{DS}} {α}_{k} [ \frac{1}{2} {( \frac{{θ}_{k} ( t ) - {m}_{S , k}}{{σ}_{S , k}} )}^{2} + log \sqrt{2 π} {σ}_{S , k} ] \qquad ( 3 )$$

where L_{NS,k}(t) and L_{S,k}(t) are negative log likelihoods of the k^{th} digital signature at the time instant t pertaining to no stress and stress states, respectively, N_{DS} is the number of digital signatures employed in the algorithm to detect acute mental stress, and αₖ are the weights determined based on the symmetrized divergence values associated with all the digital signatures:

$${α}_{k} = \frac{{D}_{k}}{{\sum }_{k = 1}^{{N}_{DS}} {D}_{k}} \qquad ( 4 )$$

where Dₖ is the symmetrized divergence pertaining to the k^{th} digital signature. The rationale underlying the above choice of the weights is to assign more weight to the digital signatures with larger separation of cohort-level probability density functions, which potentially promotes more discriminative digital signatures in the inference of acute mental stress state, i.e., p(t). Second, we developed the confidence level of p(t) as follows:

$$c ( t ) = \{ \begin{matrix}\frac{p ( t ) - \overline{p}}{1 - \overline{p}} , & p ( t ) > \overline{p} \\ \frac{\overline{p} - p ( t )}{\overline{p}} , & p ( t ) < \overline{p}\end{matrix} \qquad ( 5 )$$

where \tilde{p}=0.5 is the threshold probability level to determine no stress (p(t)<\tilde{p}) vs stress (p(t)>\tilde{p}) states. Here, c(t) is a measure of the margin p(t) has with respect to \tilde{p} (hence, the more margin, the higher confidence of inference).

Evaluation: We evaluated the proposed algorithm in a leave-one-subject-out analysis setting as follows. For each test participant, we calculated the symmetrized divergences pertaining to all the prominent digital signatures using a priori probability density distributions derived using the dataset of the remaining 23 participants. Using the symmetrized divergence values, we calculated the weights αₖ's in Eq. (4). Then, we inputted the time series sequence of the selected prominent digital signatures pertaining to the test participant on a sample-by-sample basis to their respective a priori probability density distributions pertaining to no stress vs stress states to calculate L_{NS}(t) and L_{S}(t) in Eq. (3). Then, we calculated the probability of acute mental stress p(t) in Eq. (2) and its confidence level c(t) in Eq. (5). We performed the leave-one-subject-out analysis described above for each participant (i.e., 24 times in total).

To assess the adequacy of the prominent digital signatures selected and incorporated into our algorithm, we built the acute mental stress detection algorithm in FIG. 4 using (i) the prominent digital signatures; (ii) HR; (iii) SCR; (iv) HR and SCR as an example of multi-modal digital signatures without cross-integration; and (v) 6 physiologically inspired digital signatures. Then, we compared the acute mental stress detection performance of the proposed vs competing algorithms.

To assess the advantage of using the C-VI method in deriving a priori probability density functions of the digital signatures, we built the same algorithm for detecting acute mental stress in FIG. 4 with a priori probability density functions derived simply by pooling the entire dataset (pertaining to 23 participants in the leave-one-subject-out analysis setting) and calculating its mean and SD (called the “data pooling” technique hereafter). In addition, we also built an acute mental stress classifier using the rudimentary logistic regression widely used in classification tasks. In the case of logistic regression, the probability of acute mental stress was simply defined as the output of the logistic regression model (ranging between 0 and 1 by the sigmoid function). We determined the classification threshold as the optimal operating point (which maximizes AUC) on the Receiver Operating Characteristic (ROC) curve. We calculated the classification efficacy and confidence level associated with these competing algorithms. Then, we compared the acute mental stress detection performance of the proposed vs competing algorithms.

We used established classification efficacy metrics and confidence level in Eq. (5) as measures of the performance and robustness of the algorithm. We calculated the sensitivity, specificity, and accuracy of acute mental stress state classification in no stress (initial baseline and rest periods) and stress (acute mental stress intervention periods) states on a participant-by-participant basis, and summarized them across all the participants in terms of mean and SD. In addition, we likewise calculated the confidence level of acute mental stress inference in no stress and stress states on a sample-by-sample basis in each participant, aggregated them into mean value on a participant-by-participant basis, and summarized them across all the participants in terms of mean and SD.

To examine the statistical significance associated with the difference in the classification efficacy and the confidence level between (i) the proposed algorithm armed with various digital signatures (Table 1 and Table 2) and (ii) 3 competing algorithms armed with the optimal number of prominent digital signatures (Table 3 and Table 4), we used the Wilcoxon signed rank test with Bonferroni correction for multiple comparisons.

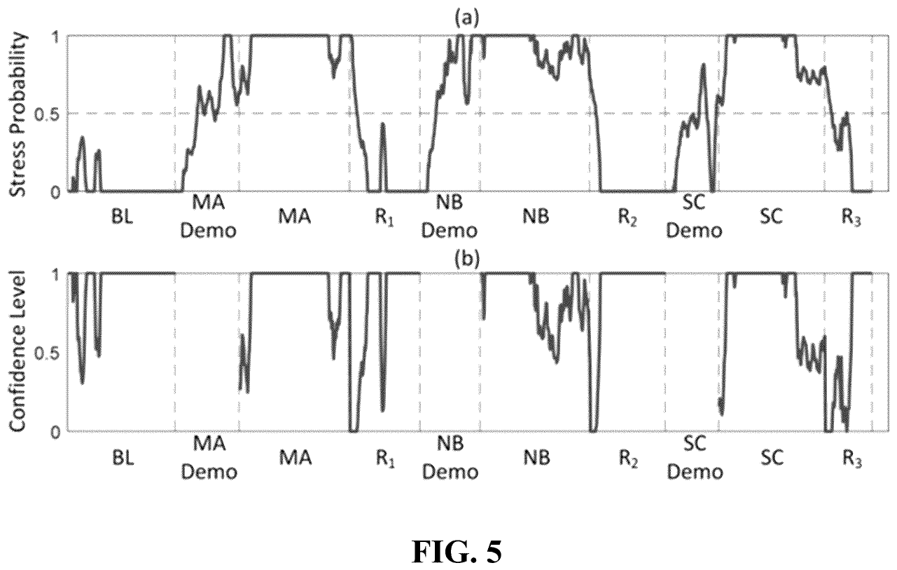

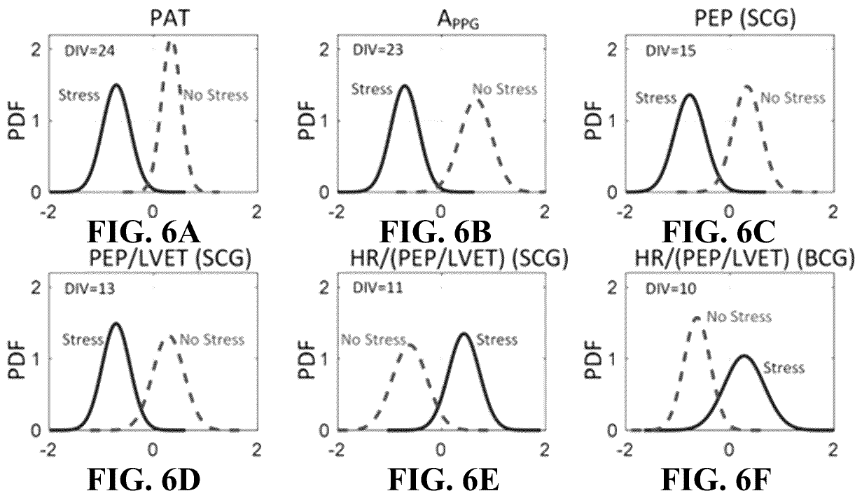

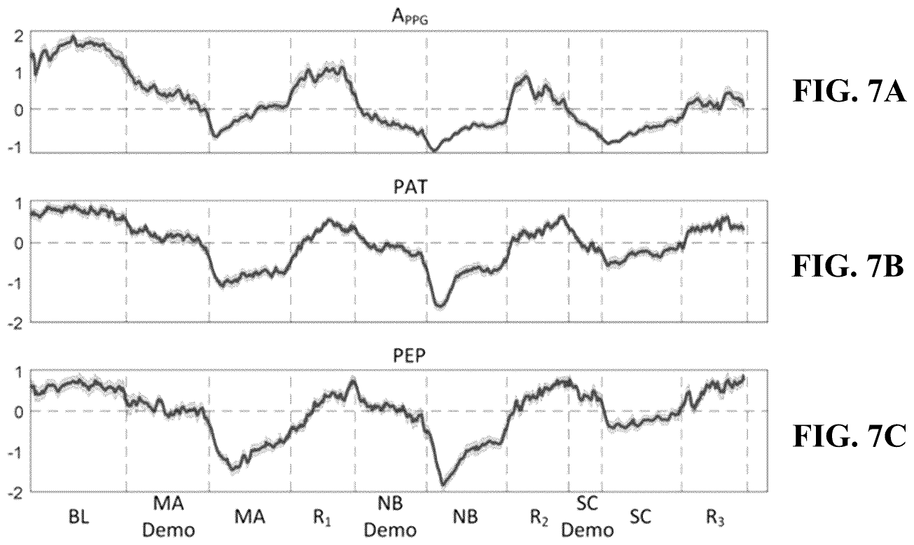

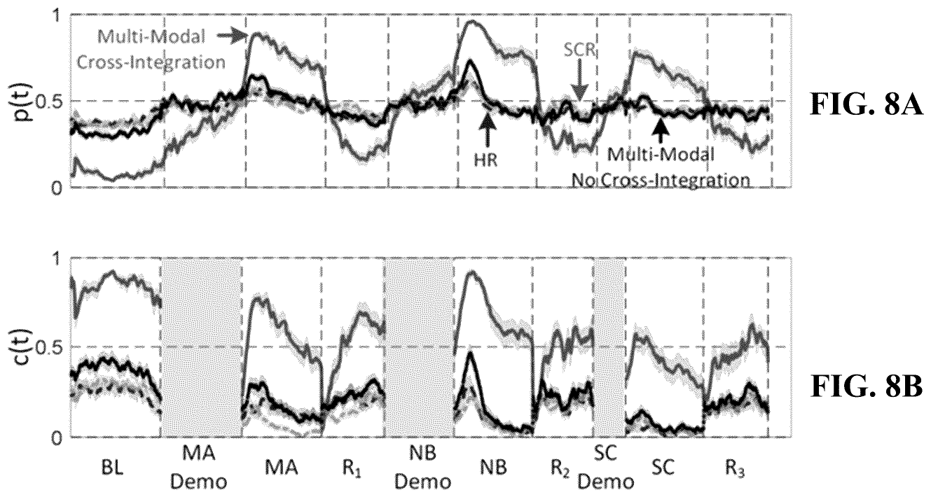

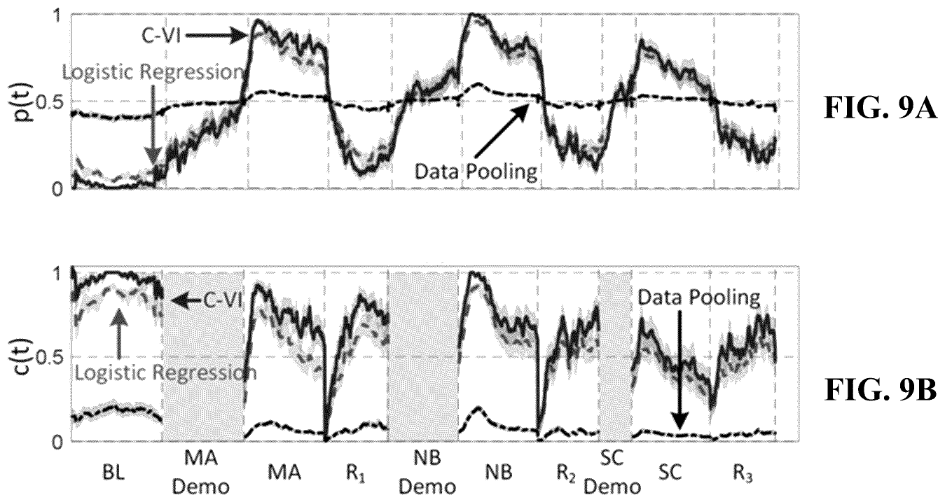

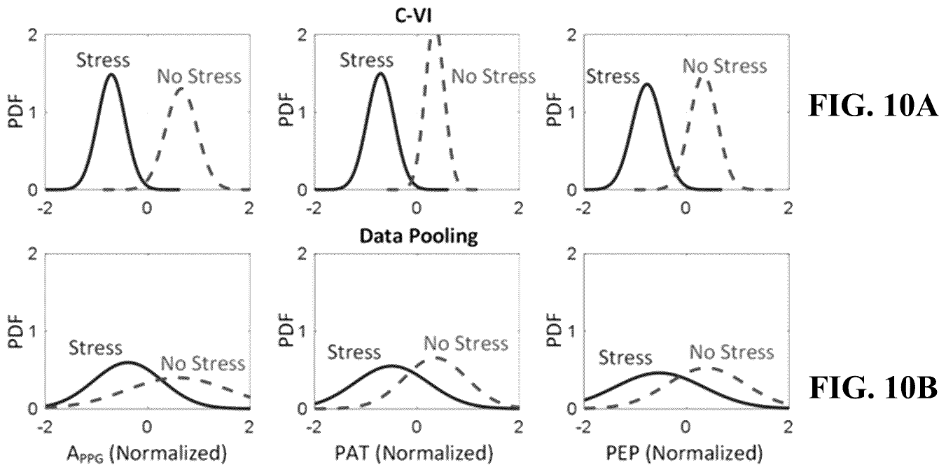

Table 1 and Table 2 summarize and compare the acute mental stress classification efficacy and the corresponding confidence level, respectively, pertaining to the proposed algorithm built upon (i) 3 prominent signatures selected in this work via the symmetrized divergence values (PPG amplitude, PAT, and PEP), (ii) HR, (iii) SCR, (iv) multi-modal signatures without cross-integration (HR and SCR (both NOR and MOR)), and (v) 6 physiologically-inspired signatures (DP, SP, SV, TPR, HR, and CO). FIG. 5 shows a representative example of dynamic changes in (a) mental stress probability and (b) the corresponding confidence level in a participant, predicted by the proposed algorithm based on the 3 prominent digital signatures. FIG. 6 shows the probability density distributions of top 6 candidate digital signatures pertaining to stress (blue solid) and no stress (red dashed) states. FIG. 7 shows the cohort-averaged dynamic changes in the prominent digital signatures, namely: (a) PPG amplitude, (b) PAT, and (c) PEP. FIG. 8 shows the cohort-averaged dynamic changes in (a) mental stress probability and (b) the corresponding confidence level inferred by (i) cross-integrated multi-modal digital signatures (PPG amplitude, PAT, and PEP), (ii) conventional univariate digital signatures (HR and SCR), and (iii) multi-modal digital signatures without cross-integration (HR and SCR (both NOR and MOR)). Table 3 and Table 4 summarize the acute mental stress classification efficacy and the corresponding confidence level pertaining to the proposed algorithm with a priori probability density functions of the prominent digital signatures inferred by (i) the C-VI method and (ii) naïve data pooling, as well as (iii) logistic regression, all based on the 3 prominent digital signatures. FIG. 9 shows the cohort-averaged dynamic changes in (a) mental stress probability and (b) the corresponding confidence level pertaining to the proposed algorithm with a priori probability density functions of the prominent digital signatures inferred by (i) the C-VI method and (ii) naïve data pooling, as well as (iii) logistic regression, all based on the 3 prominent digital signatures. FIG. 10 shows the probability densities of 3 prominent digital signatures associated with the C-VI method vs naïve data polling technique.

TABLE 1
Stress state classification efficacy.

| | Sensitivity | Specificity | Accuracy |
|---|---|---|---|
| DIV-Based Prominent | 0.85+/−0.12 | 0.86+/−0.09 | 0.85+/−0.07 |
| Signatures | | | |
| Heart Rate | 0.65+/−0.22 | 0.59+/−0.17* | 0.61+/−0.18* |
| Skin Conductance Response | 0.67+/−0.19* | 0.63+/−0.13* | 0.64+/−0.12* |
| Multi-Modal, No Cross- | 0.73+/−0.17* | 0.60+/−0.14* | 0.65+/−0.11* |
| Integration | | | |
| Physiologically-Inspired | 0.84+/−0.12 | 0.86+/−0.09 | 0.85+/−0.08 |
| Signatures | | | |

*p < 0.012 relative to DIV-based prominent signatures.
DIV: symmetrized divergence.

TABLE 2
Confidence level.

| | No Stress | Stress | Combined |
|---|---|---|---|
| DIV-Based Prominent | 0.69+/−0.13 | 0.85+/−0.11 | 0.76+/−0.09 |
| Signatures | | | |
| Heart Rate | 0.07+/−0.03* | 0.38+/−0.21* | 0.21+/−0.10* |
| Skin Conductance Response | 0.11+/−0.04* | 0.31+/−0.14* | 0.20+/−0.07* |
| Multi-Modal, No Cross- | 0.08+/−0.02* | 0.32+/−0.10* | 0.18+/−0.05* |
| Integration | | | |
| Physiologically-Inspired | 0.63+/−0.12* | 0.68+/−0.14* | 0.65+/−0.11* |
| Signatures | | | |

*p < 0.012 relative to DIV-based prominent signatures.

Despite the long-standing interest in the detection of acute mental stress using digital signatures derived from physiological signals, prior work has predominantly focused on the use of a small number of digital signatures of acute mental stress derived disjointly from individual physiological signals. In this work, we demonstrated the preliminary proof-of-concept of acute mental stress detection based on novel digital signatures derived from cross-integration of multiple modalities of physiological signals. In this section, we summarize major findings and limitations.

Multi-Modal Physiological Sensing-Enabled Mental Stress Detection: Feasibility: Acute mental stress may be detected via digital signatures of physiological parameters which can be derived from wearable-enabled multi-modal physiological sensing (Table 1, Table 2, FIG. 5). The example algorithm was able to infer the probability of acute mental stress continuously through time with high confidence level (FIG. 5). Its overall efficacy of discriminating stress vs no stress states was indicated by its classification accuracy, sensitivity, and specificity characteristics (Table 1) as well as confidence level associated with the classification (Table 2).

Notably, the results of our experiments were achieved with a relatively small number of interpretable digital signatures, namely, PPG amplitude representing vasomotor tone, PAT representing BP, and PEP representing cardiac contractility. In the context of our dataset, these 3 digital signatures were associated with the largest symmetrized divergence values (FIG. 6), meaning that they are the signatures which exhibit the largest separations in probability density functions between no stress vs stress states. Of course, as discussed above, there may be alternative sets of mental stress signatures depending upon data sets and other considerations.

The three digital signatures we tracked most closely in our experiments showed physiological and interpretable changes in response to acute mental stress: PPG amplitude decreased likely due to vasoconstriction; PAT decreased likely due to an increase in BP; and PEP decreased likely due to an increase in cardiac contractility (FIG. 7). In addition, these digital signatures are constructed from various physiological signals: PPG amplitude involves PPG, PAT involves ECG and PPG, and PEP involves ECG and SCG, which confirms the power of multi-modal physiological sensing and cross-integration. It is also noted that (i) these digital signatures were consistently powerful across all the participants: PPG amplitude, PAT, and PEP were selected in 100%, 100%, and 83% of the participants; and that (ii) the efficacy of the example algorithm largely improved as the number of digital signatures used increased from 1 to 3, beyond which its efficacy improved only marginally, and eventually plateaued with additional digital signatures. All in all, the results clearly suggest the feasibility of continuous detection of acute mental stress via wearable-enabled physiological sensing and novel inference algorithms.

In the context of our dataset, the example algorithm outperformed univariate signatures as well as physiologically-inspired signatures which can comprehensively characterize acute mental stress state (which include signatures of DP, SP, SV, TPR, HR, and CO) (Table 1, Table 2, FIG. 8). For context, the accuracy characteristics associated with HR and SCR were much lower than the 3 prominent digital signatures used in this work (Table 1). Thus, the inventors made the novel discovery that cross-integration of multi-modal physiological signals can result in digital signatures much more relevant to the detection of acute mental stress than those derived from individual physiological signals. Indeed, among the top 10 digital signatures found in the leave-one-subject-out analysis (in the order of accuracy), 9 were derived from cross-integration while only 1 (PPG amplitude) was derived from an individual physiological signal (top 6 signatures shown in FIG. 6). In contrast, even a subset of physiologically-inspired digital signatures (especially HR and DP) may not change sufficiently large enough in response to acute mental stress relative to the others. Indeed, ECG-based HR, SCG-based PTT, and BCG-based PTT ranked 17^{th}, 7^{th}, and 12^{th} in terms of symmetrized divergence, and likewise ranked 17^{th}, 6^{th}, and 11^{th} in terms of accuracy in the leave-one-subject-out analysis.

From these experiments, we confirmed the techniques and approaches underlying the various embodiments described herein. Thus, we established that these processes and systems make it possible to leverage highly effective and interpretable composite/multi-modal digital signatures to accurately identify, characterize, and/or track acute mental stress states.

We also determined that advanced machine learning and inference (e.g., the C-VI method) can be utilized in processes for custom development of inference models for detecting acute mental stress. In particular, we showed that the way in which we developed our example algorithm outperformed an algorithm based on the probability density functions derived from naïve data pooling and rudimentary yet widely used logistic regression in terms of the confidence level associated with the stress probability while maintaining comparable classification efficacy (Table 3, Table 4, FIG. 9). First, the probability density functions of the digital signatures of acute mental stress derived by the C-VI method was notably sharper relative to the naïve data pooling (FIG. 10). Second, the superiority of the proposed algorithm to the popular logistic regression implies the complex dependence of the decision hyperplane to discriminate stress vs no stress states on the digital signatures of acute mental stress. Noting that confidence level can be viewed as the margin from the neutral decision (i.e., equal 50% chance of stress and no stress states), the sharper probability density functions derived by the C-VI method improve the robustness of acute mental stress detection by reducing the overlap between the probability density functions pertaining to stress vs no stress states. In addition, we also speculate that the nonlinear decision hyperplane employed in the proposed algorithm (i.e., Eq. (2) and Eq. (3)) likewise improves the robustness of acute mental stress detection by capturing the complex decision hyperplane better as well as exploiting the likelihood values corresponding to stress and no stress states independently.

What is claimed is:

1. A system for identifying mental stress in a user, comprising:
a first sensor having at least one electrode configured to contact the user to measure heart-based electrical signals of the user;
a second sensor having at least one light emitter and at least one light sensor, configured to measure photoplethysmogram (PPG) signals of the user;
a third sensor having at least one force sensor, configured to measure movement signals of the user's body reflecting cardio-mechanical activity of the user; and
a processor configured to:
receive the heart-based electrical signals, the PPG signals, and the movement signals in real time via at least one electrical input of the system;
continuously determine a set of mental stress signatures, wherein at least one of the mental stress signatures is a composite signature determined from fiducial values derived from at least two of: the heart-based electrical signals, the PPG signals, and the movement signals;
provide the mental stress signatures to an inference model configured to determine a probability of an acute mental stress state of the user based on a probability density distribution associated with the mental stress signatures; and
generate an output indicative of whether the user is presently in an acute mental stress state based on the probability.

2. The system of claim 1, wherein the third sensor is a ballistocardiograph (BCG) sensor.

3. The system of claim 1, wherein the third sensor is a seismocardiography (SCG) sensor.

4. The system of claim 1, wherein the composite signature comprises at least one of a photoplethysmogram (PPG) amplitude signal, a pulse arrival time (PAT) signal, or a pre-ejection period (PEP) signal.

5. The system of claim 1, wherein the composite signature is a PAT signal that is determined based on a time between an R-wave peak in the heart-based electrical signals and foot of PPG waveform in a corresponding pulse cycle.

6. The system of claim 1, wherein the composite signature is a PEP signal that is based on a time between an R-wave peak determined from the heart-based electrical signals and a mechanical opening of the subject's aortic valve determined from the movement signals.

7. The system of claim 1, wherein the mental stress signatures comprise PPG amplitude.

8. The system of claim 1, wherein the inference model was trained via a collective variational inference process to determine a priori probability density distributions for each mental stress signature across a training data set comprising labeled periods of stress and no-stress states of subjects.

9. The system of claim 1, wherein the inference model determines the probability of acute mental stress state by determining a likelihood-of-stress value for each mental stress signature, and weighting the likelihood-of-stress values proportionally to divergence scores associated with each mental stress signature.

10. A method for tracking a user's mental stress state, comprising:
(i) obtaining physiological data regarding the user at multiple points during a measurement period, the physiological data including data types comprising at least cardio-electrical data, cardio-mechanical data, and vascular data;
(ii) determining at least heartbeat waveform information, heart valve opening information, and blood volume change information from the physiological data;
(iii) determining a first set of mental stress signatures for a first time slice during the measurement period, from the heartbeat waveform information, heart valve opening information, and blood volume change information, wherein at least one signature is a multi-modality mental stress signature derived from more than one data type of the physiological data;
(iv) providing the first set of mental stress signatures for the first time slice to a trained machine learning algorithm, to obtain probability indications of whether each first mental stress signature reflects a mental stress state and a no-mental stress state;
(v) determining a mental stress indication for the first time slice based on a weighted aggregation of the probability indications for each mental stress signature of mental stress state and no-mental stress state, and updating a mental stress tracker accordingly;
(vi) performing (iii)-(v) for a subsequent sets of mental stress signatures for subsequent time slices during the measurement period to obtain mental stress indications for the subsequent time slices; and
(vii) updating the mental stress tracker to account for the mental stress indications for the subsequent time slices, and outputting the updated mental stress tracker to a software application of a user device to provide the user with information relating to their mental stress states during the measurement period.

11. The method of claim 10, further comprising:
determining a signal quality characteristic for each data type of the physiological data for the first time slice;
selecting a subset of the data types of the physiological data based on their signal quality characteristics, and determining a set of possible mental stress signatures that could be determined from the subset of data types; and
determining the first set of mental stress signatures from the set of possible mental stress signatures.

12. The method of claim 11, wherein the first set of mental stress signatures comprise a photoplethysmogram (PPG) amplitude value, a pulse arrival time (PAT) value, and a pre-ejection period (PEP) value.

13. The method of claim 12, wherein the PAT value is based on a peak of the heartbeat waveform information and a foot of the vascular data.

14. The method of claim 12, wherein the PEP value is based on a peak of the heartbeat waveform information and an opening point of the heart valve opening information.

15. The method of claim 12, wherein the first set of mental stress signatures further comprises at least one of: a heart rate signal, a transient receptor potential (TRP) signal, or a pulse transit time (PTT) signal.

16. The method of claim 10, wherein the continuous physiological data is obtained using at least one of: a ballistocardiograph (BCG) sensor, a seismocardiography (SCG) sensor, or a heart-band.

17. The method of claim 10, wherein at least one of the first set of mental stress signatures is a composite signature determined from fiducial values derived from at least two data types of the continuous physiological data.
